(19) United States
(12) Patent Application Publication
(10) Pub. No.: US 2023/0165786 A1
(43) Pub. Date: Jun. 1, 2023

(54) lntraoral Cleaning Tablet

(71) Applicant: Dendisc Holdings Pty Ltd, Gold Coast Mail Centre (AU)

(72) Inventor: Frank Rees, Tweed Heads (AU)

(21) Appl. No.: 18/072,437

(22) Filed: Nov. 30, 2022

(30) Foreign Application Priority Data
Nov. 30, 2021 (AU) 2021903876

(51) Int. Cl. A61K 8/9789; A61C 17/00; A61K 8/11; A61Q 11/00; A23G 4/06; A23G 4/12

(52) U.S. Cl. CPC A61K 8/9789; A61C 17/00; A61K 8/11; A61Q 11/00; A23G 4/068; A23G 4/12; A23V 2002/00

(57) ABSTRACT

The intraoral cleaning tablet has a chewable gum to stimulate saliva and a fibrous ingestible material in the form of dried apple peel. The dried apple peel embedded within the chewable gum in order to clean the teeth. The chewable gum is plastic-free and biodegradable, comprised of ingredients such as mastic or chicle.

### BACKGROUND

### 1. Field of the Invention

The present invention relates to the dental industry and, more particularly to an intraoral cleaning tablet which is safe to ingest, and which can remove plaque and debris from the teeth, gums and the oral cavity without the use of water.

### 2. Description of Related Art

The most common way to clean teeth is with a toothbrush and toothpaste. However, it is not always convenient to use these items. Running water for rinsing the mouth is not always available.

Pfizer Inc. produces an oral care strip (sold under the Listerine trademark) which dissolves in the mouth and freshens the breath. However, these oral care strips do not clean the teeth.

Prior chewable toothpaste tablets can be used to create toothpaste in the mouth of the user when mixed with their saliva. However, a brush is required to remove debris and plaque from the teeth. A brush may not be available. Moreover, these chewable toothpaste tablets are not ingestible. Accordingly, if water is not available to rinse the mouth, then chewable toothpaste tablets are not practical.

Traditional chewing gums can be used to freshen the mouth, but they are not ingestible, or at least are not recommended for ingestion. Most chewing gums contain plastic, which is indigestible.

The object of the present invention is to provide an oral hygiene product which is safe to ingest, and which can remove plaque and debris from the teeth, gums and the oral cavity without the use of water.

### SUMMARY OF THE INVENTION

According to the present invention, there is provided an intraoral cleaning tablet comprising:

(a) a chewable gum to stimulate saliva; and
(b) a fibrous ingestible material;

wherein:
(i) that the fibrous ingestible material is dried apple peel embedded within the chewable gum in order to clean the teeth; and
(ii) the chewable gum is plastic-free and biodegradable.

The term ‘tablet’ can include any substance to be put into the mouth, including gums, jubes or cubes of any shape. The tablets may be coated or uncoated.

The chewable gum may include mastic gum (Pistacia lentiscus), chicle, gum Arabic, gum acacia, Chios Mastic Tree gum resin, glycerol, or waxes (such as bee's wax, carnauba wax, Candelilla wax).

The tablet may include a lubricant, such as magnesium stearate (produced from palm oil), or a combination of other ingredients such as rice extract, rice hulls, gum arabic and sunflower oil.

Preferably, the fibrous material is freeze-dried before being embedded within the chewable gum. The fibrous material may be compressed and freeze-dried.

The chewable gum may contain additives, such as calcium including hydroxyapatite, calcium phosphate (mono basic, dibasic or tribasic), calcium glycero-phosphate, calcium citrate, calcium carbonate, calcium lactate. The chewable gum may contain other additives such as sodium bicarbonate, Commiphora myrrha (myrrh) gum oleoresin powder, Piper nigrum (black pepper) essential oil, Xylitol, or hydroxyapatite.

Preferably, the tablet also contains a gelatinous substance, such as agar. The gelatinous substance may contain agents for polishing, flavouring, breath freshening and/or desensitising the teeth. The gelatinous substance may also include a health supplement, such as zinc or fluoride. The gelatinous substance may include catalytic nanozyme to reduce plaque.

The breath freshening agent may be one or more essential oils, such as mint, spearmint, peppermint, eucalyptus, Piper nigrum (black pepper) oil, citrus oils (such as lemon or orange), cloves, cinnamon, or ginger.

The tablet may include menthol as a cooling and flavouring agent.

According to another aspect of the present invention, there is provided a method of making an intraoral cleaning tablet comprising the steps of:

(a) collecting apple peel;

(b) drying the apple peel;

(c) blending the apple peel with other ingredients for the tablet to form a composition;

(d) compressing the composition; and

(e) forming the composition into a tablet.

The method may also include a step of coating the tablet in a gum. The method may also include the step of freezing the dried apple peel.

Preferably, the fibrous ingestible material is organic and contains no pesticides or herbicides.

Any of the features described herein can be combined in any combination with any one or more of the other features described herein within the scope of the invention.

### DETAILED DESCRIPTION OF THE INVENTION

The fibrous ingestible material in the form of apple peel, which is embedded within a chewable gum. The gum is preferably mastic gum but may also be chicle.

The tablet could be in the form of a hard tablet, or soft gum or jube.

The tablet can be coated or uncoated.

When the tablet is chewed, the apple peel removes the plaque and debris from the teeth, gums and oral cavity.

The apple peel is an organic material which is inherently flavoured. If the apple peel is swallowed, it would do no harm.

Chewing or sucking the tablet can help to remove plaque and stimulate saliva.

The tablet is moved around within the mouth by the tongue, cheeks and lips to clean all areas of the mouth and teeth.

The tablet contains additives, such as essential oils (including eucalyptus oil or peppermint oil). Peppermint oil is a popular essential oil used alone and in combination with other essential oils. Menthol is the main component of peppermint oil and is responsible for the noticeable cooling sensation.

The tablet contains calcium hydroxyapatite, which is useful for tooth health.

The tablet may include health supplements, such as zinc, fluoride, and Vitamin C, prebiotics, probiotics, postbiotics and herbal ingredients.

The tablet may also include catalytic nanozyme to reduce plaque.

The intraoral cleaning tablet is made by:

(a) collecting apple peel;

(b) drying the apple peel;

(c) blending the apple peel with other ingredients for the tablet to form a composition;

(d) compressing the composition; and

(e) forming the composition into a tablet.

The method preferably includes the step of freezing the dried apple peel. The method may include a step of coating the tablet in a gum.

The present invention provides an oral hygiene product which is safe to ingest because it contains organic ingredients that are plastic-free and biodegradable, which can remove plaque and debris from the teeth, gums and the oral cavity without the use of water.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. An intraoral cleaning tablet comprising:
(a) a chewable gum to stimulate saliva; and
(b) a fibrous ingestible material,
wherein:
(i) the fibrous ingestible material is dried apple peel embedded within the chewable gum in order to clean the teeth; and
(ii) the chewable gum is plastic-free and biodegradable.

2. The tablet of claim 1, wherein the apple peel is freeze dried.

3. The tablet of claim 1, wherein the chewable gum is mastic.

4. The tablet of claim 1, wherein the chewable gum is chicle.

5. The tablet of claim 1, wherein the tablet further includes a gelatinous substance encasing the chewable gum.

6. The tablet of claim 1, wherein the tablet further includes a supplement.

7. A method of making an intraoral cleaning tablet comprising the steps of:
(a) collecting an apple peel;
(b) drying the apple peel;
(c) blending the apple peel with other ingredients for the tablet to form a composition;
(d) compressing the composition; and
(e) forming the composition into a tablet.

8. The method of claim 7, wherein the method further includes the step of: freezing the dried apple peel.

9. The method of claim 7, wherein the method further includes the step of: coating the tablet in a gum.

10. The method of claim 7, wherein the other ingredients include a chewable gum that is plastic-free and biodegradable.
